(19) United States
(12) Patent Application Publication
(10) Pub. No.: US 2004/0120902 A1
(43) Pub. Date: Jun. 24, 2004

(54) Fluoride compatible calcium carbonate

(76) Inventors: Patrick Clinton Wernett, Upper Black Eddy, PA (US); Ali Reza Bashey, Bethlehem, PA (US)

Correspondence Address: Marvin J Powell, Intellectual Property, 1 Highland Avenue, Bethlehem, PA, 18017, US

(21) Appl. No.: 10/475,192

(22) PCT Filed: Apr. 24, 2001
(86) PCT No.: PCT/US01/13056

(51) Int. Cl. A61K 7/18

(52) U.S. Cl. 424/52

(57) ABSTRACT

The invention relates to a composition having finely divided calcium carbonate particles treated with fatty acids or polysaccharides. The invention further relates to a method for the preparation of the composition and its use in applications where fluoride compatibility is desired. The method entails providing finely divided calcium carbonate particles, treating the particles with at least one of fatty acids or polysaccharides including gums, starches and/or mucilages, and adding the treated particles to a fluoride containing toothpaste formulation.

### FIELD OF THE INVENTION

This invention relates to ingestable calcium carbonate compositions, a method for its preparation, and the use of such in food products, supplements, mouth washes, dentifrices, gels, chewable tablets and the like.

More particularly, this invention relates to treated calcium carbonate materials that resolves the matter of taste and interaction with other components. One particular problem addressed is that of chalky taste in food products and dietary supplements. Another problem is food system compatibility and another is the problem of fluoride stability in dental hygiene compositions.

### BACKGROUND OF THE INVENTION

Calcium carbonate is used in food products and dietary supplements as well as personal care products, which are used in the mouth. Such use can be in toothpaste and powders, dietary or nutritional supplements, antacids, food products, such as breakfast and snack foods, and the like. The benefits of such use can involve the uptake of calcium into the body system for use elsewhere and for the physical properties of the calcium carbonate in the oral cavity, such as in dental abrasives and carriers.

When calcium carbonate is present in the oral cavity as a component of such materials, the user can experience the unfavorable taste property of chalkiness. Such taste experience may be affected by the physical character of the calcium carbonate material, such as size and surface property. Another factor may be the chemical property such as ionization and the like.

Calcium carbonate as a cleaning abrasive is a commonly used component in toothpaste and tooth powders. Another component in toothpaste and tooth powders can be fluoride to enhance enamel protection. Calcium carbonate can react with fluoride to form calcium fluoride (CaF₂). When this reaction occurs, fluoride is unavailable to interact with teeth to provide protection. This interaction may occur during the use of the dental hygiene process or during storage over time. In either event, such reaction decreases the effectiveness of the fluoride treatment and is not desirable.

European Patent Application No. 0 219 483 discloses a treated calcium carbonate abrasive comprising pulverized calcium carbonate in a liquid dispersion with an alkali metal pyrophosphate to produce a pyrophosphate derivative selected from the group consisting of calcium pyrophosphate, calcium alkali metal pyrophosphate, and mixtures thereof to chemisorb on the surface of the calcium carbonate particles.

U.S. Pat. No. 4,357,318 discloses a dentifrice comprising a water soluble monofluorophosphate salt as a source of soluble fluoride in a therapeutically effective anti-caries concentration, an effective abrasive amount of calcium carbonate and a dibasic alkali metal phosphate, said dentifrice being devoid of benzyl alcohol.

U.S. Pat. No. 3, 930, 305 discloses a dental cream containing an abrasive system comprising sodium bicarbonate in a vehicle containing water and sufficient viscous water miscible polyol humectant or mixtures thereof and a sufficient amount of gelling or thickening agent to impart to the dental cream the pasty consistency, body and the non tacky nature which is characteristic of conventional dental creams or toothpastes, and a water-insoluble dental abrasive material compatible with said bicarbonate in the dental cream, said sodium bicarbonate being primarily in the undissolved solid state, said dental cream having a granular textured appearance comprising dispersed non-crystalline appearing granulate of macroscopic crystalline bicarbonate granules in an otherwise smooth continuous matrix.

U.S. Pat. No. 5,476,647 discloses a substantially phosphate-free two component system for increased deposition of fluoride onto and into dental tissue, comprising a first component of a soluble calcium source, a soluble calcium complexing agent and a buffer and a second component containing a fluoride compound and a buffer. When the two components are combined, there results a precipitation of calcium fluoride gradually and continuously over the course of about 10 seconds to about 4 minutes.

U.S. Pat. No. 4,420,312 discloses a method for the production of an abrasive composition comprising a precipitated amorphous silicon dioxide. The abrasive composition, when incorporated into toothpaste compositions containing a fluoride therapeutic agent, provides a toothpaste composition which exhibits minimal loss of soluble fluoride upon storage at normal temperatures.

U.S. Pat. No. 5,939,051 discloses a dentifrice composition, comprising an orally acceptable dentifrice vehicle and a silica hydrogel.

U.S. Pat. No. 5,891,448 discloses a two-component system for delayed sustained precipitation of calcium fluoride onto and into dental tissue comprising a first component containing a soluble calcium source, with no more than approximately ten percent of the calcium in complexed form; a second component containing a soluble fluoride compound; and a calcium fluoride inhibitor present in either or both of components. The second component is isolated from the reaction with the first component during storage and prior to use. When the two components are combined, the inhibitor produces a delay of at least about five seconds before significant formation of calcium fluoride occurs. The level of phosphate in the system is less than the concentration needed for significant precipitation of hydroxyapatite.

U.S. Pat. No. 5,723,107 discloses a method for fluoridating teeth utilizing a semi-solid, extrudable, two component dentrifice system. The steps include preparing as a first component a semi-solid, extrudable dentifrice composition containing a fluoride ion releasable hydrolyzable complex fluoride compound in an aqueous acidic vehicle in which the fluoride compound is stable, the vehicle being free of abrasive and surfactant and containing xanthan gum as the major thickening agent and glycerin, sorbitol or mixtures thereof as the humectant, and as a second component, a semi-solid extrudable aqueous dentifrice composition containing a calcium ion releasable compound and an abrasive in an aqueous vehicle contains xanthan gum as the major thickening agent and glycerin, sorbitol or mixtures thereof as the humectant. The first and second dentifrice compounds are kept separate from the other until application to teeth requiring fluoridation. Then the first and second components are mixed together to deposit calcium fluoride therefrom on contact with a tooth surface.

U.S. Pat. No. 5,145,668 discloses a method for fluoridating teeth with a reactive, multi-component composition. There are mixed a first component comprising calcium chloride together with a second component comprising sodium fluorosilicate, an acetate salt, and a sufficient quantity of soluble, non-toxic phosphorous salt to maintain the phosphorous concentration at a desired level. The sodium fluorosilicate of the reactive multi-component composition is hydrolyzed, and calcium fluoride is precipitated from the reactive multi-component composition. The reactive multi-component composition is applied to tooth surfaces for a period of time ranging from about 10 seconds to about 4 minutes.

There remains the need to provide calcium carbonate particles in dental hygiene material, food products, antacids, dietary and nutritional supplements and the like in a form which has pleasant texture and palatable taste and without interfering with other beneficial components.

An object of the present invention is to produce a calcium carbonate particle that is stable when used in an environment where fluoride ions are present (being “stable” means the calcium carbonate does not react significantly with the fluoride ions or other components).

Another object of the present invention is to provide a process for producing calcium carbonate particles that are stable in an environment where fluoride ions are present. A further object of the present invention is to provide calcium carbonate particles that are stable when used in formulations where fluoride ions are present. These and other objects of the present invention are more fully disclosed in the detailed description of the embodiments of the invention, which hereinafter follows.

### SUMMARY OF THE INVENTION

The present invention solves the problem of fluoride stability in toothpaste when calcium carbonate is used as an abrasive. In the present invention, calcium carbonate is treated with polymers and/or fatty acids making it fluoride compatible in toothpaste formulations.

The present invention is a fluoride compatible surface treated calcium carbonate dental abrasive that is effective when used in an environment where fluoride compatibility is required.

The present invention also solves the problem of chalkiness taste when ingesting material containing calcium carbonate particles by providing a calcium carbonate particle treated with polymers and/or fatty acids.

The present invention also provides enhanced shelf life to material using the treated calcium carbonate particles in that such particles have reduced reactivity with other components.

### DETAILED DESCRIPTION OF THE INVENTION

The present invention relates to a surface treated calcium carbonate particle in which the treatment is effective when the calcium carbonate particle is used in an environment where oral hygiene or ingestion is required. In particular, the surface treated calcium carbonate particular manifests beneficial taste characteristics or interacts beneficially with fluoride delivery systems by having reduced characteristics to impact fluoride stability in such systems.

One embodiment of the present invention is an ingestible material comprising calcium carbonate particles, in which the calcium carbonate particles have been effectively treated with one or more agents selected from the group consisting of fatty acids and polysaccharides to reduce the sensation of chalkiness in said ingested material due to the presence of the calcium carbonate particles.

In such use the ingestible material can be any material for consumption, such as food products, dietary and nutritional supplements, pharmaceuticals and the like. The presence of a calcium carbonate particle in such material can be for any of a variety of reasons. Such reasons include, without limitations, use of calcium carbonate to provide elemental, texture, filler or other purposes. Examples include, without limitation, processed foods, dietary and nutritional supplements, dental hygiene compositions, denture adhesives and the like.

Suitable calcium carbonate particles that may be surface treated by the process of the present invention include calcium carbonate particles having the morphological forms of aragonite, calcite, vaterite, amorphous and mixtures thereof. The calcium carbonate particles may also be synthetically produced precipitated calcium carbonate (PCC) or ground natural calcium carbonate. A preferred calcium carbonate particle has a median particle size of from about 0.5 to about 30 micrometers, preferably from about 1 to about 15 micrometers. The specific surface area of calcium carbonate particle of the present invention is from about 0.5 meters square per gram to about 50 meters square per gram. A preferred specific surface area is from about 1 to about 10 meters square per gram. The specific surface area of the calcium carbonate is defined herein as the area per unit mass based on the sorption of nitrogen using the BET method.

Suitable surface treating agents include fatty acids and polysaccharides. The fatty acids that are useful in the present invention have the general chemical formula of CH₃(CH₂)_{X}CO₂H, in which X ranges from about 2 to about 20, more preferably from about 8 to about 20. The fatty acid can be saturated or unsaturated. Substitution is permissible as long as the substitution does not substantially impact the beneficial properties of the present invention. Preferred fatty acids have the common names of lauric, palmitic, stearic, oleic, linoleic, and behenic. The treating level for fatty acids can range from about 0.01 percent to about 20 percent, preferably from about 0.05 percent to about 4 percent, based on the dry weight of calcium carbonate. The treating level can be affected by the surface area of the calcium carbonate in that as the particle size decreases, the treating level increases.

Preferred polysaccharides used in the present invention have nine or more units of monosaccharides (C₆H₁₂O₆) linked by glycosidic bonds and include, but are not limited to, gums, starches and mucilages. Other preferred polysaccharides that are useful in the present invention may be selected from the group consisting of alginates, xanthans, guar, carrageenan, gellan, and the like. The treating level for polysaccharides can range from about 0.05 percent to about 20 percent, preferably from about 0.05 percent to about 4 percent, based on the dry weight of calcium carbonate. The treating level is affected by the surface area of the calcium carbonate in that as the particle size decreases, the treating level increases generally.

One preferred polysaccharide form is starch. Starches that are useful in the present invention may be selected from the group consisting of potato, corn, tapioca, carboxymethylcellulose, and the like. The treating level for starches can range from about 0.05 percent to about 20 percent, preferably from about 0.05 percent to about 4 percent, based on the dry weight of calcium carbonate. The treating level is affected by the surface area of the calcium carbonate. What this means, is that as the calcium carbonate particle size increases the treating level decreases. As the particle size decreases, the treating level increases.

Another preferred form of polysaccharides is mucilages. Mucilages that are useful in the present invention may be selected from the group consisting of agar, tragacanth mucilage, yellow or white mustard mucilages and the like. The treating level for mucilages can range from about 0.05 percent to about 20 percent, preferably from about 0.05 percent to about 4 percent, based on the dry weight of calcium carbonate. The treating level is affected by the surface area of the calcium carbonate. What this means, is that as the calcium carbonate particle size increases the treating level decreases. As the particle size decreases, the treating level increases.

In order to treat the calcium carbonate of the present invention with a fatty acid such as, for example, stearic acid, the fatty acids can be applied to calcium carbonate by dry coating. Dry coating is achieved by adding stearic acid to a dry calcium carbonate and mixing at a temperature from about 40 to about 200 degrees Celsius. The temperature range should be sufficient enough to melt the fatty acid. The resultant calcium carbonate is treated with the fatty acid. The calcium carbonate of the present invention so treated is particularly suitable for use in mouthwashes, dentifrices, gels, and chewable tablets where fluoride compatibility is desired.

An alternative method to dry coating is to wet coat the calcium carbonate. Wet coating is achieved by adding a solution or emulsion of fatty acids or polysaccharides including gums, starches and mucilages to a calcium carbonate slurry. The calcium carbonate slurry is prepared by synthesizing calcium carbonate in an aqueous environment or adding water to a dried calcium carbonate powder.

Another alternative method for treating the calcium carbonate is by adding dry calcium carbonate to a solution or emulsion of fatty acids or polysaccharides including gums, starches and mucilages. Still another alternative method for treating the calcium carbonate is by adding dry fatty acids or polysaccharides including gums, starches and mucilages to a calcium carbonate slurry.

The calcium carbonate abrasive treated in accordance with this invention improves fluoride compatibility when incorporated into oral hygiene products such as, toothpaste, toothpowder, chewable gum, tablets, and other dentifrices.

The treated calcium carbonate abrasive of this invention can be used as the sole abrasive in the oral hygiene product or can be used in conjunction with other dental abrasives. Other suitable abrasives include water-insoluble sodium or potassium metaphosphates, hydrated or anhydrous dicalcium phosphate, sodium bicarbonate, calcium pyrophosphate, various forms of silica, zirconium, silicate and the like.

The total amount of abrasives employed in oral hygiene products can range from less than 5 percent to more than 95 percent by weight of the dentifrice. Generally, toothpaste contains from 20 percent to 60 percent by weight of abrasive. Abrasive average particle size preferably ranges from about 2 microns to 20 microns.

In addition to the abrasive, toothpaste and tooth powder compositions conventionally contain one of or a combination of a fluoride compound, sudsing agents, binders, humectants, flavoring agents, sweetening agents and water.

Suitable fluoride compounds can be any of the compounds previously mentioned conventionally employed to provide available fluoride ion in the oral cavity. Sodium monofluorophosphate, sodium fluoride and the like, have been employed with good results in toothpaste to promote dental hygiene. Good results can be achieved employing an amount of fluoride compound to provide available fluoride ion in the range of 300 to 2000 ppm in the toothpaste, preferably 1000 ppm.

Suitable sudsing agents are generally anionic organic synthetic detergents active throughout a wide pH range. Representative of such sudsing agents used in the range of about 0.5 percent to 5 percent by weight of the composition are water-soluble salts of C₁₀-C₁₈ alkyl sulfates, such as sodium lauryl sulfate; of sulfonated monoglycerides of fatty acids, such as sodium monoglyceride sulfonates; of fatty acid amides of taurine, such as sodium N-methyl-N-palmitoyltauride; and of fatty acid esters of isethionic acid, and aliphatic acylamides, such as sodium N-lauroyl sarcosinate.

Suitable binders or thickening agents to provide the desired consistency are, for example, hydroxyethylcellulose, sodium carboxymethylcellulose, natural gums, such as gum karaya, gum arabic, gum tragacanth, colloidal silicates and finely divided silica. Generally, from 0.5 percent to 5 percent by weight of the composition can be used.

Various humectants can be used, such as glycerine, sorbitol and other polyhydric alcohols.

Suitable flavoring agents include oil of wintergreen, oil of spearmint, oil of peppermint, oil of clove, oil of sassafras and the like. Saccharin, aspartame, dextrose, levulose can be used as sweetening agents.

The following examples are being presented to further illustrate and support the novelty of the present invention. They are presented for illustrative purposes only and should in no way be used to limit the scope of the coverage, which is more specifically defined by the claims that are attached hereto.

### TEST METHODS AND PROCEDURES

### XPS Surface Analysis

XPS (X-ray Photoelectron Spectroscopy) also referred to as ESCA (Electron Spectroscopy for Chemical Analysis) is a surface sensitive technique with an analysis depth of 5-50 Angstroms (Å) (0.0005-0.005 microns). Samples are bombarded with X-rays causing electrons to be emitted. The electrons which evolve without energy loss originate from the top few monolayers. The spectrometer separates these electrons according to their kinetic energy. The energies of the photoelectrons depend not only on the chemical element from which the electrons originate but also upon the chemical environments of that element. The results are expressed in atom percent. For example, if the analysis results read “1.5 percent F (NaF)”, then that is interpreted as 1.5 percent of the atoms on the surface are fluorine ions that are attached to a sodium atom.

### Hefferren Method for Assessment of Dentifrice Abrasivity

The Hefferren method measures the Radioactive Dentin Abrasivity (RDA) number, also called the Abrasivity Index (AI). This method utilizes the roots (dentin) of extracted human teeth, which are irradiated by a neutron flux. The teeth are mounted and brushed with a slurry of the toothpaste in question at a certain pressure and number of strokes. After brushing an aliquot of the slurry is dried and beta radiation is measured. The more abrasive the toothpaste under test, the more radioactivity associated with the slurry. The results are compared to a reference abrasive provided by the American Dental Association (ADA). The results are expressed as Abrasivity Index (AI). The AI values are interpreted for toothpaste as follows:

| | |
|---|---|
| Less than 99 | low abrasion |
| 100-199 | medium abrasion |
| 200-250 | high abrasion |
| greater than 250 | unacceptable |

### EXAMPLE 1

### Uncoated Calcium Carbonate vs. Coated Calcium Carbonate

Samples of uncoated and treated ground calcium carbonate products having a surface area of 1.1 meters square per gram and a median particle size of 9.1 microns were mixed in a beaker at 18 percent solids while a sodium monofluorophosphate (MFP) fluoride solution was added to a level of 0.88 percent. The slurries were allowed to mix for ten (10) minutes before filtration. The filter cakes were dried in an oven of 110 degrees Celsius overnight and surface analyzed by XPS (x-ray Photoelectron Spectroscopy).

Surface fluoride analysis results are presented in Table 1. In Table 1, F (CaF₂) denotes fluoride that has reacted with CaCO₃ and is unstable. The F (MFP) denotes fluoride that has not reacted with calcium carbonate and is therefore stable. This form of fluoride would be available to react with and protect teeth.

TABLE 1
Surface Fluorine Content of Dried Filter Cakes (atom percent)

| | | | | | 0.5% |
|---|---|---|---|---|---|
| | untreated | 0.1% Na St | 0.5% Na St | 0.1% Guar | Guar |
| F (CaF₂) | 1.5 | 0.4 | — | 1.1 | 0.8 |
| F (MFP) | — | — | — | 0.9 | 1.5 |

Note: — indicates an undetectable amount. A typical detection limit for fluoride is approximately 0.05 atom present.

XPS analyses demonstrate that coating GCC with sodium stearate (NaSt) results in a stable system.

Coating with guar gum creates a stable system. When the level of guar gum is increased from 0.1 percent to 0.5 percent, based on the dry weight of the guar and the dry weight of the calcium carbonate, the stability of the system increases.

### EXAMPLE 2

### Comparative Stability of Calcium Carbonate Using Other Treatments

The same laboratory experiments were conducted with ground calcium carbonate (GCC) as in Example 1. Each treatment was applied at a level of 0.1 percent and 0.5 percent based on dry weight of the treatment and dry weight of calcium carbonate. The results of the surface fluoride analysis of the dried filter cakes are presented in Table 2:

TABLE 2
Surface Fluoride Content

| | | F (CaF₂) | F (MFP) |
|---|---|---|---|
| Untreated GCC | | 1.5 | — |
| CMC | 0.1% | 0.7 | — |
| CMC | 0.5% | 0.9 | — |
| Carrageenan | 0.1% | 1.0 | — |
| Carrageenan | 0.5% | 1.2 | — |
| Sodium Alginate | 0.1% | 1.6 | — |
| Sodium Alginate | 0.5% | 0.3 | — |
| Xanthan | 0.1% | 0.6 | — |
| Xanthan | 0.5% | failed | — |
| Gellan | 0.1% | 1.2 | — |
| Gellan | 0.5% | 1.1 | — |
| Linoleic | 0.1% | 0.4 | — |
| Linoleic | 0.5% | 0.4 | — |
| Hydroxystearic | 0.1% | 0.5 | — |
| Hydroxystearic | 0.5% | 0.3 | — |

Table 2 demonstrates that the calcium carbonate treated with the fatty acids, linoleic and hydroxystearic, and sodium alginate, at the higher treatment levels, increase stability. The polysaccharides, xanthan, and carboxymethylcellulose also increase stability but not as well as the fatty acids and the sodium alginate.

### EXAMPLE 3

### Effects of Treatments on Abrasivity in a Toothpaste Formulation

Four experimental GCC products treated with sodium stearate and guar gum at 0.1 percent and 0.5 percent were incorporated into a toothpaste formulation with the following composition:

| | % W/W |
|---|---|
| Calcium Carbonate | 18.00 |
| Sident 22S | 11.20 |
| Titanium Dioxide | 0.50 |
| Sodium Saccharin | 0.20 |
| Sodium Benzoate | 2.20 |
| Glycerin | 12.00 |
| Sorbitol | 15.00 |
| Methyl Paraben | 0.03 |
| Xanthan Gum | 0.40 |
| Sodium Lauryl Sulfate | 1.30 |
| PEG-8 | 7.00 |
| Oil of peppermint | 0.80 |
| Purified water | 31.37 |

Five toothpaste formulations containing treated GCC abrasives were tested for abrasivity.

| Sample Description | Abrasivity Index |
|---|---|
| uncoated GCC | 136 |
| GCC + 0.5 percent stearic acid | 128 |
| GCC + 0.1 percent stearic acid | 120 |
| GCC + 0.1 percent guar gum | 163 |
| GCC + 0.5 percent guar gum | 165 |

An abrasion index for toothpaste of:

99 and less—low abrasion

100-199 medium

200-250 high

>250 unacceptable high abrasion

Stearic acid and guar gum treated GCC are in the medium abrasive range. The treatment level on the calcium carbonate does not adversely affect abrasion.

What is claimed is:

1. Composition comprising an orally acceptable hygiene media, calcium carbonate particles suitable for use as a dental abrasive, a fluoride compound suitable to provide beneficial fluoride treatment to teeth, wherein said calcium carbonate particles have been effectively treated with one or more agents selected from the group consisting of fatty acids and polysaccharides to inhibit reaction of fluoride ions of said fluoride compound and said calcium carbonate particles.

2. Composition of claim 1 wherein said media is toothpaste or tooth powder.

3. Composition of claim 2 wherein said calcium carbonate particles have an average particle size of 0.5 to 30 micrometers.

4. Composition of claim 2 wherein said calcium carbonate particles have a specific surface area of 0.5 to 50 meters squared per gram.

5. Composition of claim 1 wherein said calcium carbonate particles are precipitated calcium carbonate

6. Composition of claim 1 wherein said fluoride compound is a sodium monofluorophosphate.

7. Composition of claim 1 wherein said agent is one or more of a fatty acid selected from the group consisting of lauric acid, palmitic acid, stearic acid, oleic acid, linoleic acid, and behenic acid.

8. Composition of claim 1 wherein said agent is one or more of a polysaccharide selected from the group consisting of alginates, xanthans, guar, carrageenan, and gellan.

9. Composition of claim 1 wherein said treatment is effective to reduce the uptake of fluoride ions by said calcium carbonate material by 50 percent.

10. Composition of claim 1 wherein said treatment comprise coating at least a portion of said calcium carbonate material

11. A composition comprising an orally acceptable hygiene media, calcium carbonate particles suitable for use as a dental abrasive, a fluoride compound suitable to provide fluoride ions for beneficial treatment to teeth, and one or more agent(s) selected from the group consisting of fatty acids and polysaccharides, wherein said agent(s), said calcium carbonate particles and said fluoride ions effectively interact to create a system to inhibit reaction of said fluoride ions and said calcium carbonate particles.

12. A method of treating teeth with fluorine in an oral environment containing calcium carbonate material comprising first treating the calcium carbonate material with an agent to inhibit the uptake of fluorine by said calcium carbonate material.

13. A composition comprising an orally acceptable ingestable media and calcium carbonate particles suitable for use in said media, said calcium carbonate particles effectively coated with a polysaccharide or a fatty acid wherein said coating is effective to increase the palatability of said calcium carbonate to the ingester of said media.

14. A toothpaste composition comprising calcium carbonate particles suitable for use as a dental abrasive, a fluoride compound suitable to provide beneficial fluoride treatment to teeth, wherein said calcium carbonate particles have been effectively treated with one or more agents selected from the group consisting of fatty acids and polysaccharides to inhibit reaction of fluoride ions of said fluoride compound and said calcium carbonate particles.
